(19) United States
(12) Patent Application Publication
(10) Pub. No.: US 2009/0011458 A1
(43) Pub. Date: Jan. 8, 2009

(54) Method for selectively staining microorganisms

(76) Inventor: Paul E. Johnson, Laramie, WY (US)

Correspondence Address: JENNIFER L. BALES, MOUNTAIN VIEW PLAZA, 1520 EUCLID CIRCLE, LAFAYETTE, CO, 80026-1250, US

(21) Appl. No.: 12/214,549

(22) Filed: Jun. 19, 2008

Related U.S. Application Data
(60) Provisional application No. 60/936,234, filed on Jun. 19, 2007.

(51) Int. Cl. C12Q 1/06; C12Q 1/04

(52) U.S. Cl. 435/39; 435/34

(57) ABSTRACT

A method for selectively flagging target microorganisms in a liquid sample also including background particles comprises the steps of adding a lysing agent selected to breach the background particles to the sample, adding a dye selected to flag the target microorganisms to the sample, adding a suppressing agent selected to penetrate the breached background particles and suppress the dye within the breached background particles to the sample, and measuring the flagged target microorganisms in the sample. The method is particularly useful in organic samples such as dairy and blood product solutions.

This application claims priority to provisional application for patent No. 60/936,234 filed Jun. 19, 2007.

### BACKGROUND OF THE INVENTION

1. Field of the Invention

The present invention relates to a method for selectively staining target microorganisms in a translucent fluid for their detection. In particular, the present invention relates to methods for fluorescently staining target microorganisms in a fluid containing leukocytes, such as blood products or dairy products.

2. Description of the Related Art

Imaging and classification of low concentrations of selected target particles, cells in particular, in large volumes of fluid has a number of applications including: 1) bioterrorism and biowarfare defense, 2) food and water quality control, 3) clinical detection of cancerous cells, and 4) environmental monitoring. Cell imaging and classification systems developed to date usually suffer from 1) high cost, 2) unsatisfactory sensitivity, 3) slowness, 4) large size, 5) insufficient spectral and/or spatial resolution, and/or 6) labor-intensive preparation steps.

Direct detection may be accomplished using flow cytometry. Flow cytometry is a commonly used technique to measure the chemical or physical properties of cells. Cells flow by a measuring apparatus in single file while suspended in a fluid, usually air or water. In immunofluorescence flow cytometry, cells can be identified by attaching fluorescent antibodies to each cell:
An antibody specific to the cell of interest is labeled with a fluorescent molecule or fluorochrome.
The labeled antibody is mixed in solution with the cell of interest. The antibodies attach to specific sites on the cells (called antigens).
The cells are passed in single file in a stream of liquid past a laser(s), which illuminates the fluorochromes and causes them to fluoresce at a different wavelength.
A photomultiplier or photodiode is used to detect a burst of fluorescence emission each time a marked cell passes in front of the detector.
The number of marked cells can then be counted. Antibodies can be chosen that are highly-specific to the cell(s) of interest.

Flow cytometry is currently used for a wide variety of applications including: measuring helper T-lymphocyte counts to monitor HIV treatment, measuring tumor cell DNA content in determining cancer treatment, and separating X- and Y-chromosome bearing sperm for animal breeding.

FIG. 1 (prior art) shows a typical flow cytometry system (from Shapiro, Practical Flow Cytometry, 2nd Edition). Putting flow cytometry into practice involves using two concentric cylindrical streams of fluid. The inner flow or core flow contains the cells to be sampled. The purpose of the outer stream or sheath flow is to reduce the diameter of the core flow. As the core and sheath fluids reach the tapered region of the flow, the cross-sectional area of the core flow is reduced. A small bore core flow (about 20 microns) allows for precision photometric measurements of cells in the flow, illuminated by a small diameter laser beam; all of the cells will pass through nearly the same part of the beam and will be equally illuminated. Why not just pass the cells through a small-bore transparent tube? Small diameter orifices are generally unworkable because they experience frequent clogging. Commercial flow cytometers typically use a sheath/core flow arrangement.

Laser-induced fluorescence of fluorescent labels in a flow cytometer is a uniquely powerful method of making fast, reliable, and relatively unambiguous detections of specific microorganisms, such as food-borne pathogens. Several monographs describe the methods and applications of flow cytometry (e.g., Flow Cytometry: First Principles by A. L. Givan, 1992, and references therein). Historically, flow cytometers have been very large, expensive, laboratory-based instruments. They consume large amounts of power, and use complex electronics. They are not typically considered within the realm of portable devices. The size (desktop at the smallest), power requirements, and susceptibility to clogging (requiring operator intervention) of conventional flow cytometers precludes their use for many applications, such as field monitoring of water biocontamination.

U.S. Pat. No. 6,309,886,“High throughput analysis of samples in flowing liquid,” by Ambrose et al. discloses an invention for the high throughput analysis of fluorescently labeled DNA in a transparent medium. This invention is a device that detects cells in a flow moving toward an imaging device. The flow is in a transparent tube illuminated in the focal plane from the side by a laser with a highly elongated beam. Although this invention does not suffer from the drawbacks listed above for alternative technologies, it is not suitable for applications where the flow medium is not transparent. It is also not an imaging technology, but rather a technology suitable for single-point photometric detection and characterization.

A precursor invention, described in U.S. Pat. No. 6,765,656 by the present inventor, is shown in FIGS. 2 and 3. This invention, called Fountain Flow™ cytometry, allows detection of cells rapidly, sensitively, inexpensively, and at low concentrations in a portable device.

In FIG. 2 (Prior Art), a sample of fluorescently tagged cells 210 flows up the tube 206 toward the CCD camera and foreoptics 208. The cells are illuminated in the focal plane by a laser 228 through transparent end element 220. When the cell(s) pass through the CCD camera focal plane 234 they are imaged by the CCD camera 218 and lens assembly 212, through a transparent window and a filter 214 that isolates the wavelength of fluorescent emission. The fluid in which the cells are suspended then passes by the window and out the drain tube 230.

In FIG. 3 (Prior Art), a flow block 322 is used with a device like that shown in FIG. 1. FIG. 3A is a side schematic drawing of the aluminum flow block. FIG. 3B is a top plan view of the flow black. FIG. 3C shows a detail of the device flow and imaging. The sample enters the flow block through Tygon tube 312 and stainless steel tube 310 and exits through stainless steel tube 324 and Tygon tube 315. Two 2-mm holes have been drilled into the aluminum flow block 322, an entrance hole 302 and an exit hole 306. As the sample flows up the internal entrance hole 302, it passes through the focal plane of the CCD camera 326. This hole is generally painted black to reduce scattered light. Component 320 is a Teflon tape gasket. The gasket is sandwiched between the aluminum flow block and a circular window 220, and tightly held with a screw-on brass cap 318. The gasket is cut to form a channel 304 through which the fluid is diverted into the exit hole 306. FIG. 3D is a photograph of a working flow block with attached tubing. The block is mounted onto a black-anodized plate.

There are many and various clinical and industrial applications requiring the detection and/or enumeration of microorganisms in various background matrices (e.g. food, beverages, and body fluids) for quality control and clinical diagnoses. It is common practice to use a dye specific to certain types of microorganisms so that it is easy to contrast cells of interest against a background of other particles, including other kinds of cell/microorganisms.

This is particularly important in techniques such as Fountain Flow™ cytometry or conventional flow cytometry whereby an automated method is used to detect and count cells of interest based on their fluorescent intensity. In these cases it is common to use one of a variety of stains that are specific to the microorganism of interest, for example an immunolabel, where fluorescent molecules are attached to an antibody specific to the microorganism of interest. Even then, however, background particles in the fluid can absorb the “specific” dye and produce false detections. Background particles can also autofluoresce at the same wavelength as the fluorescent label. With this in mind, it is desirable to produce a method that stains only the target particles of interest, introduces no or little fluorescence anywhere else in the samples of interest, and suppresses fluorescence in the background and background particles.

### SUMMARY

An object of the present invention is to provide a method that stains only the target particles of interest and introduces no or little fluorescence anywhere else in the samples of interest. An invention is described which allows fluorescence measurements of specific, potentially pathogenic, target microorganisms in a fluid sample, in particular blood, blood products, milk, and milk products. The invention described here is a dye combination, or cocktail that separates target microorganisms (such as from background interference, especially fluorescent emission from white blood cells, or leukocytes such as those found in milk or blood, that have absorbed the primary stain).

A method according to the present invention for selectively flagging target microorganisms in a liquid sample also including background particles comprises the steps of adding a lysing agent selected to breach the background particles in the sample, adding a dye selected to flag the target microorganisms in the sample, adding a suppressing agent selected to penetrate the breached background particles and suppress the dye within the breached background particles in the sample as well as suppressing background fluorescence from the liquid, and measuring the flagged target microorganisms in the sample.

Depending upon the sample, the dye might comprise a cell wall permeable intercalating DNA dye, a cell wall permeable yeast-specific dye, a yeast viability dye, or a chitin dye. For example, the following dyes are useful in this process: SYTO-13, SYTO-16, picoGreen, FUN1, FUN2, and Solophenyl Flavine.

Examples of suppressing agent are: propidium iodide, Trypan Blue, Evans Blue, and Crystal Violet. A combination of propidium iodide and Trypan Blue works well.

The lysing agent might comprise comprises detergent, distilled water, or saline. The step of measuring the flagged target microorganisms may utilize a Fountain Flow™ cytometer to enumerate the target microorganisms.

A kit for selectively staining microorganisms generally includes at least one of each of the following: 1) a primary dye to which the intact cell wall of a living target organism is permeable, 2) a secondary dye (generally called a “suppressant” herein to distinguish it from the primary dye) which prevents the primary dye from fluorescing wherever the two coexist and at the same time is not cell-wall permeable for target cells with intact cell walls, and 3) a cell lysing agent which, in a specific range of concentrations, will breach cell walls of leukocytes and not the cell wall of target microorganisms. The kit may also include a buffer such as sodium citrate or tri acetate EDTA. If the lysing agent is distilled water or saline, it may not be necessary to include it in the kit.

As a feature, the translucent sample may be diluted to render it sufficiently transparent for Fountain Flow™ Cytometry shown in FIGS. 2-3C (Prior Art). In some cases the diluent and lysing agent are one and the same.

### BRIEF DESCRIPTION OF THE DRAWINGS

FIG. 1 (Prior Art) is a schematic drawing showing a conventional flow cytometry system.

FIG. 2 (Prior Art) is a simplified schematic drawing showing apparatus for detecting particles in a translucent flow according to a precursor of the present invention.

FIG. 3A (Prior Art) shows a side cutaway view of a flow block that may be used in the apparatus of FIG. 2. FIG. 3B (Prior Art) is a top plan view of the flow block. FIG. 3C is a detail side view of the flow block illustrating depth of focus.

FIG. 4 is a flow diagram illustrating the method of selectively staining target microorganisms according to the present invention.

FIG. 5 is a flow diagram illustrating the method of the present invention applied to a blood product (for example, whole human blood) containing fungal cells.

FIG. 6 is a flow diagram illustrating the method of the present invention applied to a milk product (for example whole milk, 2% milk, or non-fat milk) containing bacteria.

FIG. 7 is a diagram showing a kit for selectively dying microorganisms according to the present invention.

FIG. 8 is a plot showing experimental results comparing target microorganism counts according to the present invention with plate counts

### DETAILED DESCRIPTION OF THE PREFERRED EMBODIMENTS

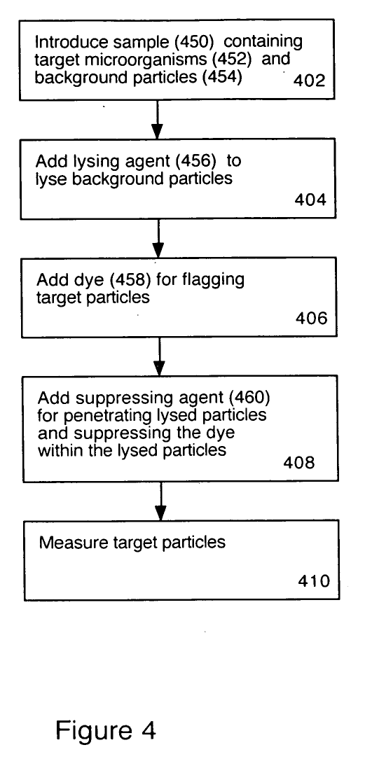

FIGS. 4-6 are flow diagrams illustrating variations on the method of the present invention. Those skilled in the art will appreciate other variations within the scope of the invention. For example, the steps may not always be performed in the order shown.

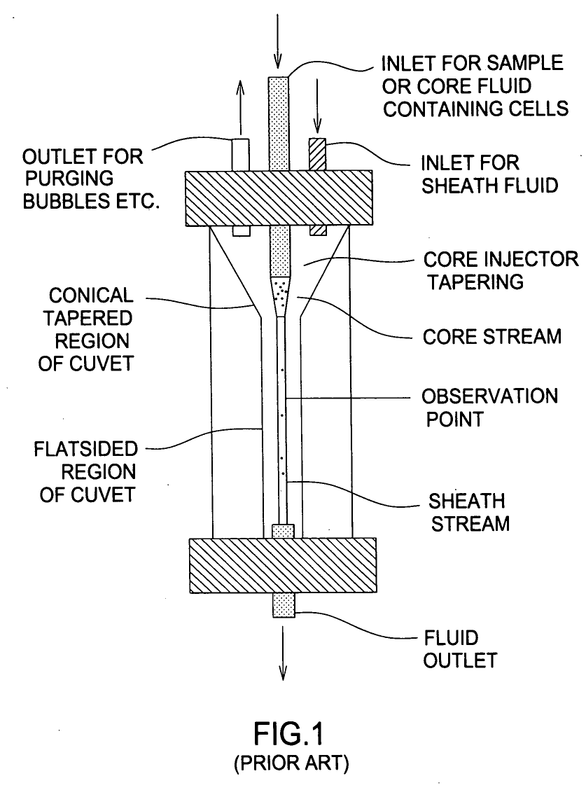

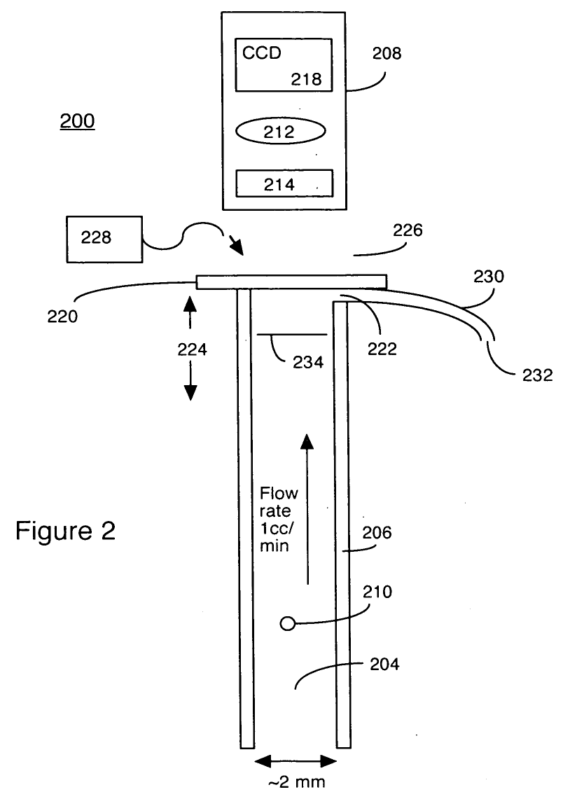

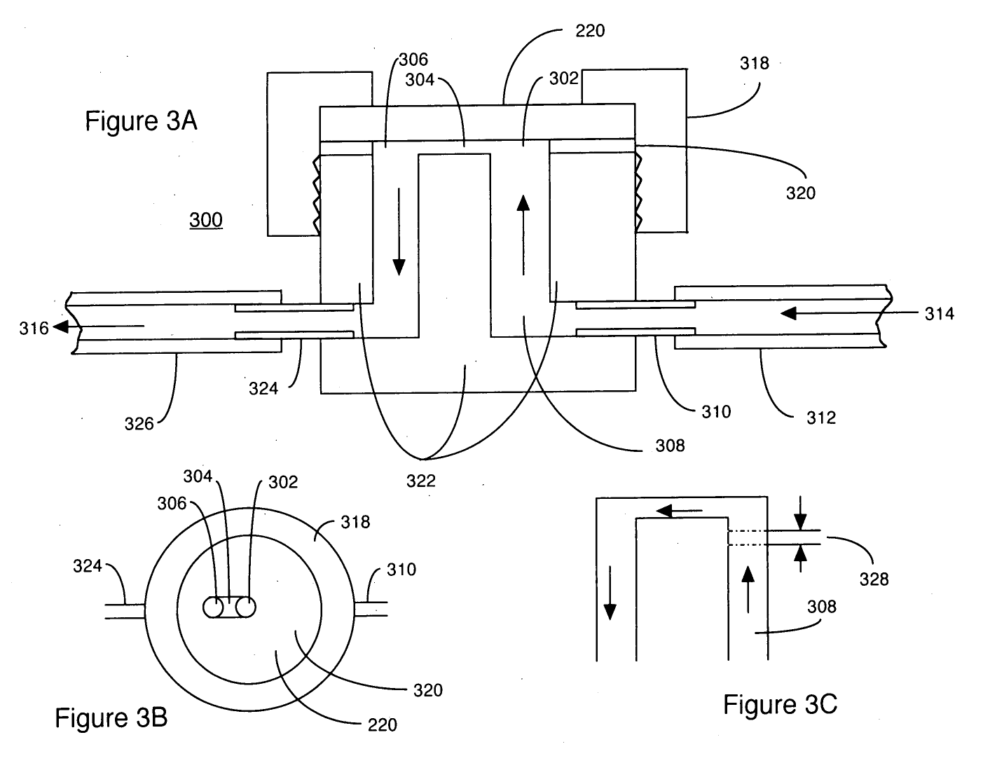

FIG. 4 is a flow diagram illustrating the method of selectively staining target microorganisms according to the present invention. In steps 402-408, a sample 450 is prepared prior to being introduced into the detection/measuring apparatus, such as the traditional cytometer of FIG. 1 (Prior Art) or the Fountain Flow™ Cytometer of FIGS. 2-3C (Prior Art). The sample includes target microorganisms 452 to be detected, and background particles 454 to be ignored. Dye 458 will flag the target microorganisms, but has a tendency to also flag (or stain) background particles, resulting in false positives. Hence a suppressant 460 is used to suppress the dye within the background particles as well as the fluid medium in general, and a lysing agent 456 lyses the background particles in order to allow the suppressant to penetrate the background particles.

In step 404 a lysing agent 456 is added to the sample to lyse the background particles. Lysing agent 456 must be carefully chosen and added at the right concentration to lyse the background particles but leave the target microorganisms unaffected. For example a detergent such as Triton X-100 diluted in distilled water might be added in high enough concentrations to breach leukocytes in an organic sample, but not to penetrate the living bacteria comprising the target particles. We have found that a concentration of ˜0.25% Triton X-100 in distilled water will lyse leukocytes but leave bacteria unaffected. When detecting yeast in blood, distilled water might be added to lyse leukocytes and red blood cells, leaving yeast cells intact, while reducing the opacity of the fluid sufficiently to facilitate Fountain Flow™ cytometry.

In step 406, a dye is added to the sample 450. It is generally preferable to use a fluorescent dye, as these are most effective for detecting and enumerating target particles. The dye is chosen to flag the target microorganisms. For example, cell wall permeable intercalating DNA dyes such as SYTO 13, SYTO 16 or picoGreen (Invitrogen) are useful to flag, or stain, cellular DNA. Alternatives include cell wall permeable yeast-specific dyes such as FUN1 or FUN2 (Invitrogen), a yeast viability dye which stains intravacuolar structures in fungal cells, and Solophenyl Flavine 7GFE 500, a chitin dye which stains the cell walls of fungi. In step 408, a fluorescence suppressing agent 460 is added to the sample 450. Suppressing agent 460 prevent the primary dye from showing up outside of the target particles (prevents it from fluorescing in the case of a fluorescent dye). Since the background particles have been breached, suppressant 460 is able to enter and prevent the dye from emitting light with significant intensity from within the background particles. This prevents false positives from the background particles as well as from the fluid medium in general. At the same time, suppressant 460 is not able to penetrate the target microorganisms, as these have not been breached. Thus these target microorganisms will be flagged. The suppressant might comprise a single substance or a combination (cocktail) of substances. For example one or more of the following might be used: propidium iodide, Trypan Blue, Evans Blue, and Crystal Violet. Propidium iodide and Trypan Blue, in particular have been used by us to suppress fluorescence from background cells (including white blood cells) dyed with fluorescent DNA dyes (e.g. the Invitrogen SYTO dyes and picoGreen) in milk, human whole blood, and blood platelets. In addition, we have used propidium iodide and Trypan Blue, to suppress background from background cells in blood when staining Candida albicans (yeast) with a chitinous dye, Solophenyl Flavine.

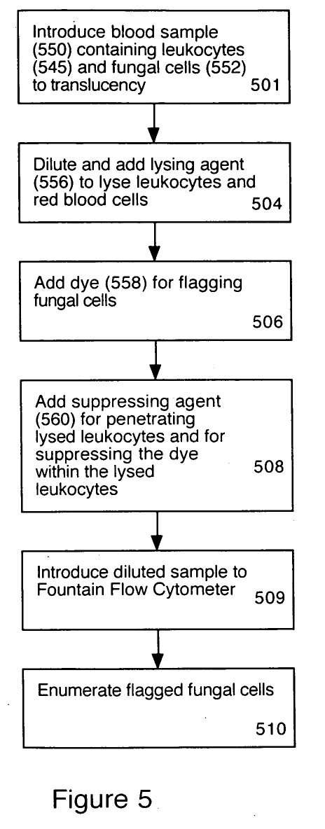

FIG. 5 is a flow diagram illustrating a specific example of the method of the present invention applied to a human whole blood sample 550 containing fungal cells 552 (for example Candida albicans). In this example, sample 550 is diluted in step 501 in order to render it translucent, for use in a Fountain Flow Cytometer. This dilution will also lyse cells (red blood cells in particular). For example, in step 506, a dye 558 such as FUN1 or Solophenyl Flavine is added to dye the fungal cells. In step 508, suppressing agent 560, for example Trypan Blue, is added to the sample, in order to prevent the leukocytes from fluorescing. In one experiment, 2 mg of Trypan Blue was added to a 10 ml sample of diluted whole blood.

In step 509, the sample is introduced into the test equipment. In this example, the samples were loaded into a 3 ml syringe and passed through a Fountain Flow™ cytometer using a syringe pump at a rate of 1.8 ml/hr. 500 images were taken using an Electrim CCD camera and an exposure time of 0.4 ms. There was no significant elapse time between exposures so the total time spent taking images for each set was 200 seconds or 3.33 minutes. Thus 0.1 ml of fluid was passed through the cytometer during each data set.

Counting 510 was performed using the method described in U.S. Pat. No. 6,765,656 (incorporated herein by reference) to the present inventor. Plate counts on YM agar (with an 18 hour incubation) were used to confirm our Fountain Flow™ cytometry enumeration (FFC counts).

FIG. 8 shows experimental results comparing FFC counts according to the method of U.S. Pat. No. 6,765,656 to plate counts of C. albicans spiked into a 1:20 dilution of whole blood into saline, stained with a cocktail of SYTO-16, Triton X-100, Trypan Blue, and propidium iodide. The sample was further diluted 1:100 into distilled water, which lysed non-fungal cells. The line of best fit gave a 97% counting efficiency. Post-dilution concentrations of C. albicans ranged from 0 to 120 per ml.

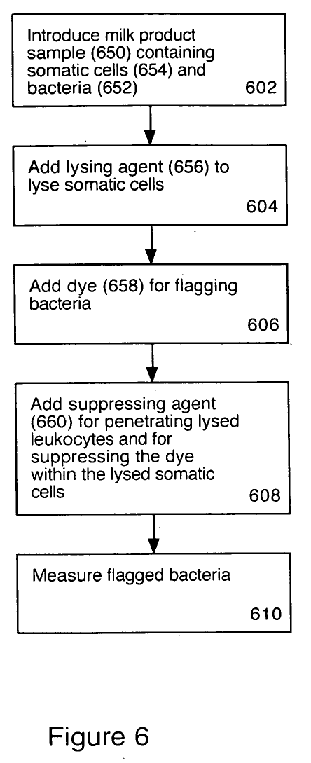

FIG. 6 is a flow diagram illustrating the method of the present invention applied to a sample 650 comprising a milk solution containing target bacteria 652 (for example E. coli) and background leukoctyes 654. In step 602, sample 650 is introduced into testing equipment. In step 604, a lysing agent 656 (for example Triton X-100 diluted in distilled water) is added to the sample in order to breach the leukocytes 654. In step 606, a dye 658 such as SYTO-16 is added to the sample to flag the bacteria. SYTO-16 is a cell membrane permeable DNA activated dye.

In step 608, suppressing agent 660 is added to the sample. In this example, propidium iodide is used to prevent the SYTO-16 from fluorescing within the breached leukocytes. Propidium iodide is a membrane impermeable dye, so it will only label microorganisms with breached membranes. Thus, leukocytes are rendered non-fluorescent or at most weakly fluorescent. This allows for measurement of the brightly fluorescing bacteria in step 610.

In an experimental setup, a set of microscope measurements were made using an Olympus BH-2 epifluorescence microscope and a FITC (fluorescein) filter set. The staining protocol was optimized to render live bacteria as easily detectable in the emission band for SYTO-16, but leave leukocytes and dead bacteria as undetectable. For a 1-ml sample of 1/100 raw milk in buffer, the following concentrations optimized the labeling (for epifluorescent microscopy) of live bacteria but render dead bacteria and leukocytes at most weakly fluorescent: 80 μl propidium iodide (of a 2.5 mg/ml solution, or 0.2 mg/ml net), 56 μl SYTO-16 (as supplied by Invitrogen), and 75 μl of 2% Triton X-100. Later in our experiments we included 0.2 mg/ml of Trypan Blue (40 μl/ml of a 5 mg/ml stock solution). The latter considerably suppressed the autofluorescent background of raw milk. We call this the standard cocktail. In addition, using sodium citrate, a relatively inexpensive buffer, as the milk diluent, increased the intensity of cell fluorescence.

Increasing the propidium iodide concentration to 10× significantly reduced false detections from background. Use of Trypan Blue in the standard cocktail reduced fluorescence outside of viable bacteria cell walls.

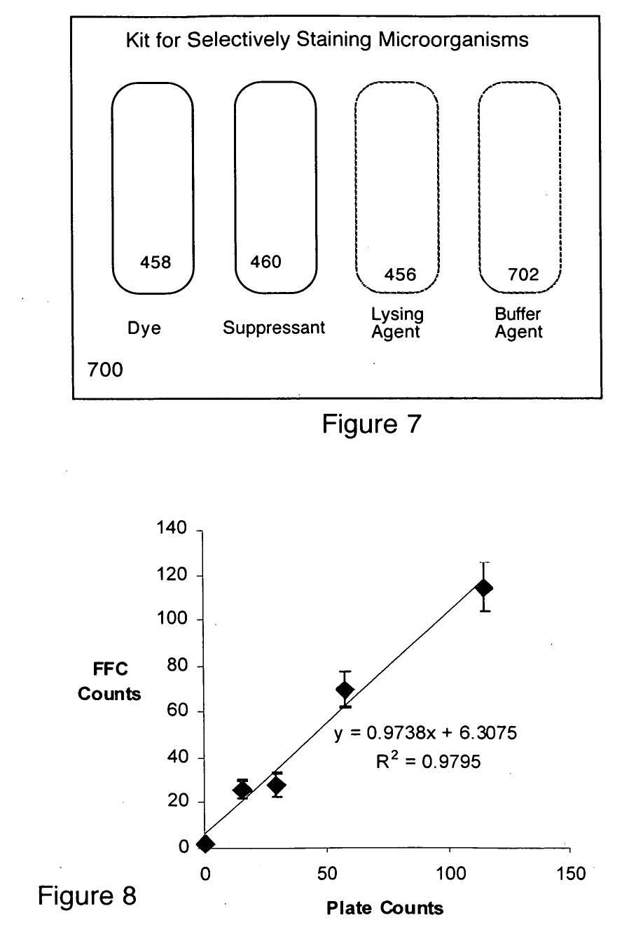

FIG. 7 is a diagram showing a kit 700 for selectively dying microorganisms according to the present invention. Kit 700 is customized to the solution 450 that is to be tested, including its target microorganisms 452 and its background particles 454. Hence, dye 458 is selected to stain target microorganisms 452, lysing agent 456 is selected to lyse the background particles 454, and suppressing agent 460 is chosen to block the effect of dye 458 in the background particles 454 and the solution itself.

As an example, kit 700 optimized for detecting E. coli in raw milk (diluted 1/100) might contain the following:

TABLE 1
Sample 450: 1% raw milk in buffer

| | |
|---|---|
| Lysing agent 456 | 75 μl at 2% Triton X-100 (per ml sample) |
| Dye 458 | 56 μl SYTO-16 (per ml sample) |
| Suppressing agent 460 | 0.2 mg propidium iodide (per ml sample) |
| | 0.2 mg Trypan Blue (per ml sample) |

As a feature, the kit could further include a buffer 702 for the sample. The choice of buffer, and its concentration relative to the other substances added to the sample can have a significant to strong effect on the degree of fluorescence. For example sodium citrate and tri acetate EDTA have proven effective in increasing fluorescence.

Note also that in some specific cases of target microorganisms and background particles, distilled water or saline can comprise the lysing agent 456. In this case, lysing agent 456 would likely not be included in the kit, but the other kit elements must of course be selected to work with the lysing agent, and the specific target microorganisms and background particles.

FIG. 8 shows experimental results comparing FFC counts according to the present invention and the enumeration method of U.S. Pat. No. 6,765,656 to plate counts of C. albicans spiked into a 1:20 dilution of whole blood into saline, stained with a cocktail of SYTO-16, Triton X-100, Trypan Blue, and propidium iodide. The sample was further diluted 1:100 into distilled water, which lysed non-fungal cells. The line of best fit gave a 97% counting efficiency. Post-dilution concentrations of C. albicans ranged from 0 to 120 per ml.

In this example the blood was diluted 1/20 in saline to form sample 450. Then 100 μl of the sample was diluted in a solution comprising 9 ml of water and 1 ml of 20% Triton X-100 (the lysing agent 456). To this was added the dye 458 comprising 100 μl of SYTO-16, and the suppressing agent 460 comprising 2 mg of propidium iodide and 2 mg of Trypan Blue. While a less dilute solution may well prove more effective, the count accuracy was impressive.

It will be appreciated by one skilled in the art that there are many possible variations on these designs that fall within the scope of the present invention.

What is claimed is:

1. The method for selectively flagging target microorganisms in a liquid sample also including background particles comprises the steps of:
(a) adding a lysing agent selected to breach the background particles in the sample;
(b) adding a dye selected to flag the target microorganisms in the sample;
(c) adding a suppressing agent selected to penetrate the breached background particles and suppress the dye within the breached background particles in the sample as well as suppressing background fluorescence from the liquid; and
(d) measuring the flagged target microorganisms in the sample.

2. The method of claim 1 wherein the dye comprises a cell wall permeable intercalating DNA dye.

3. The method of claim 1 wherein the dye comprises a cell wall permeable yeast-specific dye.

4. The method of claim 1 wherein the dye comprises a yeast viability dye.

5. The method of claim 1 wherein the dye comprises a chitin dye.

6. The method of claim 1 wherein the dye comprises at least one of the following:
SYTO-13;
SYTO-16;
picoGreen
FUN1;
FUN2;
Solophenyl Flavine.

7. The method of claim 1 wherein the suppressing agent comprises at least one of the following:
propidium iodide;
Trypan Blue;
Evans Blue;
Crystal Violet;

8. The method of claim 2 wherein the suppressing agent comprises a combination of propidium iodide and Trypan Blue.

9. The method of claim 1 wherein the lysing agent comprises detergent.

10. The method of claim 1 wherein the target microorganisms are yeast and the lysing agent is distilled water.

11. The method of claim 1 where the target microorganisms are bacteria, the liquid samples are blood platelet units, and the lysing agent is distilled water.

12. The method of claim 1 wherein the step of measuring the flagged target microorganisms utilizes a Fountain Flow™ cytometer to enumerate the target microorganisms.

13. A kit for selectively flagging target microorganisms in a liquid sample also including background particles comprising:
a dye selected to flag the target microorganisms; and
a suppressing agent selected to penetrate breached background particles and suppress the dye within the breached background particles;
wherein the kit is designed for use in combination with a lysing agent selected to breach the background particles.

14. The kit of claim 13 wherein the lysing agent comprises water.

15. The kit of claim 14 wherein the lysing agent comprises distilled water.

16. The kit of claim 14 wherein the lysing agent comprises saline solution.

17. The kit of claim 13 further including the lysing agent as part of the kit.

18. The kit of claim 13 further including a fluorescence enhancing buffer agent as part of the kit.

19. The kit of claim 18 wherein the buffer comprises sodium citrate.

20. The kit of claim 18 wherein the buffer comprises tri acetate EDTA.

21. The kit of claim 13 wherein the dye comprises a cell wall permeable intercalating DNA dye.

22. The kit of claim 21 wherein the dye comprises a cell wall permeable yeast-specific dye.

23. The kit of claim 13 wherein the dye comprises a yeast viability dye.

24. The kit of claim 13 wherein the dye comprises a chitin dye.
